(19) United States
(12) Patent Application Publication
(10) Pub. No.: US 2004/0076593 A1
(43) Pub. Date: Apr. 22, 2004

(54) Composition containing a modified nitrocellulose

(75) Inventors: Pascale Bernard, Sucy-En-Brie (FR); Jean Mondet, Aulnay-S-Bois (FR); Peyman Ghandchi, Paris (FR); Beatrice Toumi, Verrieres Le Buisson (FR)

Correspondence Address: OBLON, SPIVAK, MCCLELLAND, MAIER & NEUSTADT, P.C., 1940 DUKE STREET, ALEXANDRIA, VA, 22314, US

(73) Assignee: L'OREAL, Paris (FR)

(21) Appl. No.: 10/379,916

(22) Filed: Mar. 6, 2003

Related U.S. Application Data
(60) Provisional application No. 60/365,185, filed on Mar. 19, 2002.

(30) Foreign Application Priority Data
Mar. 8, 2002 (FR) 02 02950

(51) Int. Cl. A61K 7/04

(52) U.S. Cl. 424/61; 514/57

(57) ABSTRACT

The present invention relates to compositions, preferably cosmetic compositions, especially a varnish composition, containing at least a modified nitrocellulose. New nitrocelluloses also make up a part of the invention. A preferred embodiment is a composition containing at least one organic solvent and a modified nitrocellulose containing free hydroxyl functions partially or totally replaced with radicals —OYR, in which R represents a linear or branched hydrocarbon-based chain of 1 to 500 carbon atoms or a cyclic hydrocarbon-based chain of 3 to 500 carbon atoms, the chains being saturated or unsaturated and optionally comprising one or more O, N, S, Si and/or P atoms, and Y represents a single bond or a bonding group.

### REFERENCE TO PRIOR APPLICATIONS

This application claims priority to U.S. provisional application Ser. No. 60/365,185 filed Mar. 19, 2002, and to French patent application 02 02950 filed Mar. 8, 2002, both of which are incorporated herein by reference in their entirety.

### SUMMARY OF THE INVENTION

The present invention relates to compositions, preferably cosmetic compositions, especially a varnish composition, comprising a modified nitrocellulose. A preferred embodiment is a composition comprising at least one organic solvent and a modified nitrocellulose comprising free hydroxyl functions partially or totally replaced with radicals —OYR, in which R represents a linear or branched hydrocarbon-based chain of 1 to 500 carbon atoms or a cyclic hydrocarbon-based chain of 3 to 500 carbon atoms, said chains being saturated or unsaturated and optionally comprising one or more of O, N, S, Si and/or P atoms, and Y represents a single bond or a bonding group. In another preferred embodiment this composition can be used as a nail varnish composition. Associated methods of preparation and use of the compositions also make up a part of the invention, as do new nitrocelluloses. As noted above, the invention compositions may in a preferred embodiment be cosmetic compositions such as nail varnishes. These possibilities are denoted by reference to “(cosmetic) compositions” herein and are all included in the terms “composition” and “compositions.”

### TECHNICAL FIELD AND BRIEF DESCRIPTION OF THE PREFERRED EMBODIMENTS OF THE INVENTION

Nitrocellulose is a polymer consisting of an assembly of nitrated anhydroglucose rings partly obtained by esterification of some of the free hydroxyl functions of a cellulose with nitric acid in the presence of sulphuric acid.

Currently, nitrocellulose is still the main film-forming agent most widely used in solvent-based nail varnishes in optimized staying-power and gloss formulations. However, formulations comprising nitrocelluloses have the following drawbacks:

they produce films with a satisfactory level of hardness and gloss, but with unsatisfactory staying power over time, due especially to the poor flaking resistance of the film;

they give hard films that lack adhesion on the nail. This drawback may be overcome by adding plasticizers, but in this case very large amounts of plasticizers and co-resins, of the order of that of the nitrocellulose, need to be added. Furthermore, the presence of plasticizers in these formulations is reflected, after film formation and drying, especially by a change in the properties of the film over time, due both to the slow evaporation of the residual solvents contained in the film after drying, and to a potential loss of some of the plasticizers, especially by evaporation, resulting in hardening of the film over time and poor flaking resistance.

Investigations performed to replace nitrocellulose with other film-forming agents such as polyacrylics and polyurethanes have not given satisfactory results.

Thus, it has been envisaged to modify nitrocellulose in order to improve its properties. As result of these investigations, the inventors have discovered, surprisingly, that a suitably modified nitrocellulose can produce, preferably in a cosmetic formulation:

plasticization of films without the need to add large amounts of external plasticizers, while at the same time maintaining a good level of hardness of the films;

and/or an increase in the adhesion, for example, to the nails, of the modified nitrocellulose films and thus better flaking resistance.

These results are obtained for example with a (cosmetic) composition comprising at least one organic solvent and a nitrocellulose comprising free hydroxyl functions replaced, totally or partially, with radicals —OYR, in which R represents a linear or branched hydrocarbon-based chain of 1 to 500 carbon atoms or a cyclic hydrocarbon-based chain of 3 to 500 carbon atoms, said chains being saturated or unsaturated and optionally comprising one or more O, N, S, Si and/or P atoms, and Y represents a single bond or a bonding group.

### DETAILED DESCRIPTION OF THE PREFERRED EMBODIMENTS

According to the invention, Y representing a single bond means that O and R are directly linked by a covalent bond.

According to the invention, the hydrocarbon-based chains R are linked to the oxygen atoms of the anhydroglucose rings of the nitrocellulose, either via single bonds or via bonding groups Y, which may be chosen from —(C═O)—, —(C═O)O—, —SO₂, —CO—NH—, —CO—NR′— and —Si(R₃)₂— groups, the radicals R₃, which may be identical or different, being a linear or branched hydrocarbon-based chain of 1 to 500 carbon atoms or a cyclic hydrocarbon-based chain of 3 to 500 carbon atoms, said chains being saturated or unsaturated and possibly comprising one or more O, N, S, Si and/or P atoms and R′ denoting a C1 to C4 alkyl radicals. Preferably, R₃ comprises from 1 to 10 carbon atoms.

Preferably, in the modified nitrocellulose according to the invention, the hydrocarbon-based chains R comprise from 4 to 100 carbon atoms.

In order to reinforce the adhesion capacities of the modified nitrocellulose according to the invention, the hydrocarbon-based chains R may also comprise at least one adhesion-promoting group.

Preferably, the adhesion-promoting groups are located in the hydrocarbon-based chains R laterally and/or at the end thereof.

Preferably, these groups are present at a content of less than or equal to 10% by weight of the nitrocellulose and preferably less than or equal to 5%.

By way of example, the adhesion-promoting groups may be chosen from the following groups:

hydroxyl —OH;

carboxylic acid or ester —CO₂R₁;

chloro —Cl;

amino —NR₁R₂ with R₁ and R₂, which may be identical or different;

pyridine of formula:

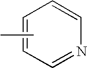

pyrimidino of formula:

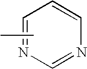

oxazolino of formulae:

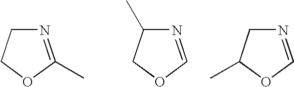

amido —NH—CO—R′ or —CO—NH—R₁;

pyrrolidono of formulae:

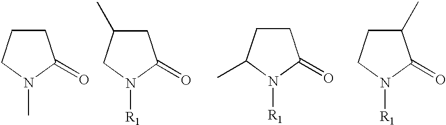

carbamoyl —O—CO—NH—R′ or —NH—CO—O—R′;

thiocarbamoyl of formula —O—CS—NHR₁ or —NH—CS—O—R′;

carbonato —O—CO—O—R′;

ureyl —NR₁—CO—N(R₁)₂, the radicals R₁ being identical or different;

thioureyl —NR₁—CS—N(R₁)₂, the radicals R₁ being identical or different;

oxamido —NR₁—CO—CO—N(R₁)₂, with the radicals R₁ being identical or different;

guanidino —NH—C(═NH)—N(R₁)₂, with the radicals R₁ being identical or different;

biguanidino —NH—C(═NH)—NH—C(═NH)—N(R₁)₂, with the radicals R₁ being identical or different;

sulphonic ester —O—S(═O)₂—R′;

sulphonamido —NR₁—S(═O)₂—R′;

acetoacetyl —O—CO—(CH₂)ₐ—CO—R′, with a denoting an integer ranging from 1 to 10 and preferably ranging from 1 to 5;

siloxane —Si(R′)_{3-b}(OR′)_{b}, with b denoting an integer ranging from 1 to 3;

acetal —OR′,

R₁ and R₂ denoting H or a C1 to C4 alkyl radical, R′ denoting a C1 to C4 alkyl radical.

Preferably, according to the invention, the degree of molar grafting of the radicals of formula OYR replacing, totally or partially, the free hydroxyl groups per anhydroglucose ring of the nitrocellulose, according to the invention, is from 0.1 to 2 and preferably from 0.25 to 0.80.

According to the invention, the hydrocarbon-based chains R grafted onto the nitrocellulose may be of polymeric and/or non-polymeric nature.

A chain of polymeric nature, according the invention, means that this chain results from the polymerisation of one or several monomers.

Particularly, when the chains are of polymeric natures, they are not, preferably, vinylic and/or acrylic chains.

As regards the physicochemical characteristics, the modified nitrocelluloses according to the invention preferably have only one glass transition temperature Tg such that Tg is less than or equal to 100° C. and preferably ranging from 30 to 70° C.

The glass transition temperature (Tg) of a polymer is measured by DMTA (Dynamic and Mechanical Temperature Analysis).

To measure the glass transition temperature (Tg) of a polymer, viscoelasticimetry tests are performed using a DMA2980 machine from T.A. Instruments, on a sample of polymer film about 200 μm thick, 5 mm wide and with a working length of about 10 mm. The sample is prepared by casting a solution of the polymer into a Teflon-coated mould and then drying on a plate thermostatically maintained at 30° C. for 7 days, under ambient humidity conditions (typically 50% RH±15%). The sample is placed under tensile stress, as small deformations (for example a sinusoidal displacement of ±8 μm is imposed thereon) at a frequency of 1 Hz during a temperature gradient ranging, for example, from −50° C. to +150° C., with a temperature variation of 3° C. per minute.

The complex modulus E*=E′+iE″ of the test polymer is then measured as a function of the temperature.

From these measurements, the dynamic moduli E′ and E″ and the damping power: tgδ=E″/E′ are deduced.

Next, the curve of the values of tgδ is plotted as a function of the temperature; this curve may show a peak. The glass transition temperature Tg of the polymer corresponds to the temperature at which the top of this peak is located (maximum of the curve). This temperature is not necessarily unique.

Preferably, the modified nitrocelluloses according to the invention are capable of forming a film that has a storage modulus E′ of greater than or equal to 100 MPa, especially ranging from 100 MPa to 5 000 MPa, preferably greater than or equal to 300 MPa, especially ranging from 300 to 1 000 MPa, and/or a damping power tgδ greater than or equal to 0.4, especially ranging from 0.4 to 1.5, preferably greater than or equal to 0.6, especially ranging from 0.6 to 1, at a temperature of 30° C. and at a frequency of 1 Hz.

Preferably, the nitrocelluloses according to the invention are capable of forming a film that has an ultimate strain εᵣ of greater than or equal to 5%, especially ranging from 5% to 500%, preferably greater than or equal to 15%, especially ranging from 15% to 400%, and/or an energy at failure per unit volume Wᵣ of greater than or equal to 0.2 J/cm³, especially ranging from 0.2 to 100 J/cm³, preferably greater than 1 J/cm³, especially ranging from 1 to 50 J/cm³.

The ultimate strain and the energy at failure per unit volume are determined by tensile tests performed on a polymer film about 200 μm thick. The film is obtained by casting a solution of the polymer into a Teflon-coated mould, followed by drying on a plate thermostatically maintained at 30° C. for 7 days, under ambient humidity conditions.

To perform these tests, the film is cut into dumbbell-shaped specimens with a working length of 33±1 mm and a working width of 6 mm. The cross section (S) of the specimen is then defined as: S=width×thickness (cm²); this cross section will be used to calculate the stress.

The tests are performed, for example, on a tensile testing machine sold under the name Lloyd® LR5K. The measurements are performed at room temperature (20° C.).

The specimens are drawn at a travelling speed of 33 mm/min, corresponding to a speed of 100% elongation per minute.

A travelling speed is thus imposed and the elongation ΔL of the specimen and the force F required to impose this elongation are simultaneously measured. From these data ΔL and F, the stress σ and strain ε parameters are determined.

A curve of stress σ=(F/S) as a function of the strain ε=(ΔL/Lₒ)×100 is thus obtained, the test being performed up the failure of the specimen, L₀ being the initial length of the specimen.

The ultimate strain εᵣ is the maximum strain of the sample before the point of failure (in %).

The energy at failure per unit volume Wr in J/cm³ is defined as the area under this stress/strain curve such that:

$${W}_{r} = {\int }_{0}^{{ϵ}_{r}} σ \cdot ϵ \cdot ⅆ ϵ$$

More particularly, the (cosmetic) compositions comprising a modified nitrocellulose as defined above may be used as a nail varnish composition for making up the nails and/or false nails, comprising in its formulation a modified nitrocellulose according to the invention, as a film-forming agent. The formulation of these varnishes may comprise pigments and nailcare active agents.

This nitrocellulose makes it possible to limit or even dispense with the content of external plasticizers in the nail varnish, thus reinforcing the adhesion and the mechanical properties of the coats to give better staying power over time.

In the abovementioned (cosmetic) compositions, the nitrocellulose content may range from 0.1% to 60% by weight, and may preferably range from 1% to 50% by weight and better still from 5% to 40% by weight, relative to the total weight of the composition.

The nail varnish composition may be used as a varnish base, as a nail makeup product, as a topcoat composition, to be applied over the nail makeup product, or alternatively as a cosmetic nailcare product. These compositions may be applied to human nails or to false nails.

The (cosmetic) compositions of the invention preferably comprise at least one organic solvent.

The organic solvents, which may be used in the (cosmetic) compositions of the invention, include the following:

ketones that are liquid at room temperature, such as methyl ethyl ketone, methyl isobutyl ketone, diisobutyl ketone, isophorone, cyclohexanone and acetone;

alcohols that are liquid at room temperature, such as ethanol, isopropanol, diacetone alcohol, 2-butoxyethanol and cyclohexanol;

glycols that are liquid at room temperature, such as ethylene glycol, propylene glycol, pentylene glycol and glycerol;

propylene glycol ethers that are liquid at room temperature, such as propylene glycol monomethyl ether, propylene glycol monomethyl ether acetate and dipropylene glycol mono-n-butyl ether;

short-chain esters (containing from 3 to 8 carbon atoms in total), such as ethyl acetate, methyl acetate, propyl acetate, n-butyl acetate and isopentyl acetate;

ethers that are liquid at room temperature, such as diethyl ether, dimethyl ether or dichlorodiethyl ether;

alkanes that are liquid at room temperature, such as decane, heptane, dodecane and cyclohexane;

cyclic aromatic compounds that are liquid at room temperature, such as toluene and xylene;

aldehydes that are liquid at room temperature, such as benzaldehyde and acetaldehyde,

mixtures thereof.

The organic solvent may preferably represent from 40 to 90% by weight relative to the total weight of the composition.

The compositions of the invention may also comprise water, especially in a content ranging from 0.1% to 10% by weight relative to the total weight of the composition, preferably less than 2% by weight of water.

The (cosmetic) composition according to the invention incorporating the above modified nitrocellulose may also comprise one or more additives chosen from additional film-forming polymers, plasticizers, dyestuffs such as pigments, nacres, flakes, thickeners, spreading agents, wetting agents, dispersants, antifoams, preserving agents, UV-screening agents, active agents, surfactants, waxes, moisturizers, fragrances, neutralizers, stabilizers and antioxidants.

Thus, the composition may comprise an additional film-forming polymer that may be chosen from free-radical polymers, polycondensates and polymers of natural origin.

The additional film-forming polymer may preferably be chosen especially from the group formed by vinyl polymers, polyurethanes, polyesters, alkyd resins, epoxyester resins, cellulose esters, for instance cellulose acetate, cellulose acetopropionate or cellulose acetobutyrate, resins resulting from the condensation of formaldehyde with an arylsulphonamide, and blends thereof.

The additional film-forming polymer may be also a nitrocellulose (not modified as defined above), which permits to obtain a nail varnish with good cosmetic properties (especially gloss properties) without having to add great quantities of plasticizers and co-resins.

The additional film-forming polymer may be present in a content ranging from 0.1% to 30% by weight and preferably ranging from 1% to 15% by weight, relative to the total weight of the composition.

The composition may also comprise at least one plasticizer. Useful plasticizers, alone or as a mixture, include:

glycols and derivatives thereof, such as diethylene glycol ethyl ether, diethylene glycol methyl ether, diethylene glycol butyl ether or diethylene glycol hexyl ether, ethylene glycol ethyl ether, ethylene glycol butyl ether and ethylene glycol hexyl ether;

glycerol esters,

propylene glycol derivatives and in particular propylene glycol phenyl ether, propylene glycol diacetate, dipropylene glycol butyl ether, tripropylene glycol butyl ether, propylene glycol methyl ether, dipropylene glycol ethyl ether, tripropylene glycol methyl ether, diethylene glycol methyl ether and propylene glycol butyl ether,

acid esters, especially carboxylic acid esters, such as citrates, phthalates, adipates, carbonates, tartrates, phosphates and sebacates,

oxyethylenated derivatives, such as oxyethylenated oils, especially plant oils such as castor oil;

mixtures thereof.

The amount of plasticizer may be chosen by a person skilled in the art on the basis of his general knowledge, so as to obtain a composition that has cosmetically acceptable properties. The plasticizer content may range, for example, from 0.1% to 15% by weight and preferably from 0.5% to 10% by weight, relative to the total weight of the composition.

The composition may comprise a dyestuff, which may be chosen from pulverulent compounds and/or colorants that are soluble in the medium of the composition. The dyestuff may be present in a content ranging from 0.001% to 10% by weight relative to the total weight of the composition. The pulverulent compounds may be chosen from the pigments and/or nacres and/or flakes usually used in nail varnishes.

The pigments may be white or coloured, and mineral and/or organic. Among the mineral pigments that may be mentioned are titanium dioxide, optionally surface-treated, zirconium oxide or cerium oxide, and also iron oxide, chromium oxide, manganese violet, ultramarine blue, chromium hydrate and ferric blue, and metallic pigments, for instance aluminium or bronze. Among the organic pigments that may be mentioned are carbon black, pigments of D & C type, lakes based on cochineal carmine, on barium, strontium, calcium or aluminium, and guanine. The nacreous pigments may be chosen from white nacreous pigments such as mica coated with titanium or with bismuth oxychloride, coloured nacreous pigments such as titanium mica with iron oxides, titanium mica with, especially, ferric blue or chromium oxide, titanium mica with an organic pigment of the abovementioned type, and also nacreous pigments based on bismuth oxychloride.

The flakes may be chosen from those made of acrylic resin, polyester, polyethylene terephthalate or of aluminium.

The colorants are, for example, Sudan red, DC Red 17, DC Green 6, β-carotene, soybean oil, Sudan brown, DC Yellow 11, DC Violet 2, DC Orange 5 or quinoline yellow.

The composition according to the invention may also comprise any additive known to those skilled in the art as being able to be incorporated into such a composition, such as thickeners, spreading agents, wetting agents, dispersants, antifoams, preserving agents, UV-screening agents, active agents, surfactants, waxes, moisturizers, fragrances, neutralizers, stabilizers and antioxidants. Needless to say, a person skilled in the art will take to select this or these optional additional compound(s), and/or the amount thereof, such that the advantageous properties of the composition for the use according to the invention are not, or are not substantially, adversely affected by the envisaged addition.

Among the modified nitrocelluloses, which are to be incorporated in the (cosmetic) compositions, some among them are new compounds especially interesting for their film-forming properties, their anti-flaking properties, their adhesion properties and their gloss qualities.

Such nitrocelluloses are nitrocelluloses comprising free hydroxyl functions replaced, totally or partially, with radicals —OYR, in which R represents a linear or branched hydrocarbon-based chain of 1 to 500 carbon atoms or a cyclic hydrocarbon-based chain of 3 to 500 carbon atoms, said chains being saturated or unsaturated and possibly comprising one or more O, N, S, Si and/or P atoms, and Y represents a single bond or a bonding group and said chain comprising at least one adhesion-promoting group chosen from the following groups:

chloro —Cl

amino —NR₁R₂ with R₁ and R₂, which may be identical or different;

pyridine of formula:

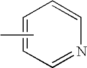

pyrimidino of formula:

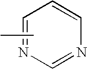

oxazolino of formulae:

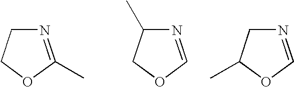

amido —NH—CO—R′ or —CO—NH—R₁;

pyrrolidono of formulae:

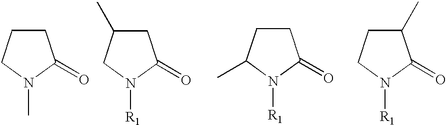

carbamoyl —O—CO—NH—R′ or —NH—CO—O—R′;

thiocarbamoyl of formula —O—CS—NHR, or —NH—CS—O—R′;

carbonato —O—CO—O—R′;

ureyl —NR₁—CO—N(R₁)₂, the radicals R₁ being identical or different;

thioureyl —NR₁—CS—N(R₁)₂, the radicals R₁ being identical or different;

oxamido —NR₁—CO—CO—N(R₁)₂, with the radicals R₁ being identical or different;

guanidino —NH—C(═NH)—N(R₁)₂, with the radicals R₁ being identical or different;

biguanidino —NH—C(═NH)—NH—C(═NH)—N(R₁)₂, with the radicals R₁ being identical or different;

sulphonic ester —O—S(═O)₂—R′;

sulphonamido —NR₁—S(═O)₂—R′;

acetoacetyl —O—CO—(CH₂)ₐ—CO—R′, with a denoting an integer ranging from 1 to 10 and preferably ranging from 1 to 5;

siloxane —Si(R′)_{3-b}(OR′)_{b}, with b denoting an integer ranging from 1 to 3;

acetal —OR′,

R₁ and R₂ denoting H or a C1 to C4 alkyl radical, R′ denoting a C1 to C4 alkyl radical.

According to the invention, the hydrocarbon-based chains R are linked to the oxygen atoms of the anhydroglucose rings of the nitrocellulose, either via single bonds or via bonding groups Y chosen from —(C═O)—, —(C═O)O—, —SO₂, —CO—NH—, —CO—NR′— and —Si(R₃)₂— groups, the radicals R₃, which may be identical or different, being a linear or branched hydrocarbon-based chain of 1 to 500 carbon atoms or a cyclic hydrocarbon-based chain of 3 to 500 carbon atoms, said chains being saturated or unsaturated and possibly comprising one or more O, N, S, Si and/or P atoms and R′ denoting a C1 to C4 alkyl radicals. Preferably, R₃ comprises from 1 to 10 carbon atoms.

Preferably, in the modified nitrocellulose according to the invention, the hydrocarbon-based chains R comprise from 4 to 100 carbon atoms.

Preferably, the adhesion-promoting groups are located in the hydrocarbon-based chains R laterally and/or at the end thereof.

Preferably, these groups are present at a content of less than or equal to 10% by weight of the nitrocellulose and preferably less than or equal to 5%.

Preferably, according to the invention, the degree of molar grafting of the radicals of formula OYR replacing, totally or partially, the free hydroxyl groups per anhydroglucose ring of the nitrocellulose, according to the invention, is from 0.1 to 2 and preferably from 0.25 to 0.80.

According to the invention, the hydrocarbon-based chains R grafted onto the nitrocellulose may be of polymeric and/or non-polymeric nature.

As regards the physicochemical characteristics, the modified nitrocelluloses according to the invention preferably have only one glass transition temperature Tg such that Tg is less than or equal to 100° C. and preferably ranging from 30 to 70° C.

Preferably, the modified nitrocelluloses according to the invention are capable of forming a film that has a storage modulus E′ of greater than or equal to 100 MPa, especially ranging from 100 MPa to 5 000 MPa, preferably greater than or equal to 300 MPa, especially ranging from 300 to 1 000 MPa, and/or a damping power tgδ greater than or equal to 0.4, especially ranging from 0.4 to 1.5, preferably greater than or equal to 0.6, especially ranging from 0.6 to 1, at a temperature of 30° C. and at a frequency of 1 Hz.

Preferably, the nitrocelluloses according to the invention are capable of forming a film that has an ultimate strain εᵣ of greater than or equal to 5%, especially ranging from 5% to 500%, preferably greater than or equal to 15%, especially ranging from 15% to 400%, and/or an energy at failure per unit volume Wᵣ of greater than or equal to 0.2 J/cm³, especially ranging from 0.2 to 100 J/cm³, preferably greater than 1 J/cm³, especially ranging from 1 to 50 J/cm³.

The nitrocelluloses of the invention may be prepared by different processes known from the man skilled in the art.

Especially, the nitrocelluloses of the invention may be prepared by grafting, in an inert solvent, hydrocarbon-based chains R as defined above onto all or some of the free hydroxyl functions of a starting nitrocellulose.

The starting nitrocelluloses used to introduce the hydrocarbon-based chains may be film-forming nitrocelluloses usually used in inks, paints and nail varnish formulations. These nitrocelluloses are prepared industrially by esterifying cellulose with a mixture of nitric acid and sulphuric acid, the latter acting as a dehydrating agent and shifting the esterification equilibrium. They thus form cellulose nitrates, commonly known as nitrocellulose.

Preferably, the starting nitrocelluloses have a percentage of nitrogen of 10% to 13.5% by weight (a fully nitrated cellulose having a percentage of nitrogen of 14.14%), which corresponds substantially to a degree of nitration of 1.7 to 2.5 esterified hydroxyl groups per anhydroglucose ring on the 3 —OH groups of the ring that are initially available for nitration. In other words, there is between 1 and 0.75 free —OH group remaining, for the grafting of hydrocarbon-based chains R, per anhydroglucose ring of the nitrocellulose.

As regards the molecular weight of the starting nitrocelluloses, it is generally expressed by measuring the (falling-ball) viscosity of a solution of the polymer (or collodion) in a mixture of solvents at a given percentage of polymer, the said percentage of polymer generally being from 12 to 25%. In general, the mixture of solvents used for the characterization consists of 25% of 95% denatured ethanol, 20% of ethyl acetate and 55% of toluene, the percentages being expressed on a weight basis relative to the mixture of solvents.

The starting nitrocelluloses used to prepare the modified nitrocelluloses according to the invention preferably have falling-ball viscosities ranging from (1/16) to 1 000 seconds and preferably from (¼) to 100 seconds, for a polymer concentration of 12.2% in the mixture of solvents mentioned above.

As regards the conditioning, the starting nitrocelluloses are, on account of their flammable nature and their explosive power, generally presented:

either in wetted form with, for example, 35% alcohol (ethanol or isopropanol);

or in solution, in a solvent free of labile hydrogen;

or in the form of “chips”, i.e. a mixture comprising, for example, 80% of the polymer and 20% of a plasticizer, such as dibutyl phthalate.

Given the hazards involved in handling the starting nitrocellulose, the processes for preparing the modified nitrocelluloses according to the invention will preferably be performed under mild conditions, i.e.

working at a reaction temperature from 0 to 80° C. and preferably from 20 to 60° C.;

working, preferably, in the absence of oxygen, to avoid any risk of explosion by contact of the nitrocellulose with an oxidizing agent, for example working under an inert gas such as argon.

The process for preparing the modified nitrocelluloses according to the invention may have several embodiments, depending on the nature of the grafted hydrocarbon-based chain.

Thus, according to a first embodiment of the process, corresponding to the case in which the hydrocarbon-based chains R are of non-polymeric origin, the grafting reaction may be carried out in an inert solvent by means of a reaction chosen from etherification, esterification reactions with a carboxylic acid or derivatives thereof, transesterification reaction with an ester or a carbonate, esterification reaction with a sulphonic acid or derivatives thereof, reaction with an isocyanate, or reaction with an alkoxysilane.

For the sections hereinbelow, in the description of the methods for preparing the modified nitrocellulose according to the invention, the following abbreviations will be used:

Nitrocell-OH for the unmodified starting nitrocellulose, only one OH being taken into account, for the purpose of clarity of the description;

R for the hydrocarbon-based chain to be grafted onto the nitrocellulose, corresponding to the formula given above, i.e. a linear or branched hydrocarbon-based chain of 1 to 500 carbon atoms, or a cyclic hydrarbon-based chain of 3 to 500 carbon atoms, said chain being saturated or unsaturated and may comprise one or more O, N, S, Si and/or P atoms, and Y is preferably from 4 to 100 carbon atoms.

By way of example, the starting nitrocellulose may be represented by the following formula:

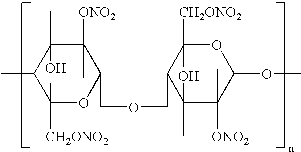

n representing the number of repeating, partially nitrated anhydroglucose dimer units in the nitrocellulose. The OH groups may be substituted with —ONO₂ groups randomly on the primary and/or secondary —OH groups of the anhydroglucose ring.

In the section hereinbelow, various reactions that may be used to graft chains R of non-polymeric nature will be described.

1) Etherification

For etherification, the following reactions may be envisaged, these being reactions for which the radical Y forming the junction between the chains R and the nitrocellulose is a single bond:

reaction between an alkyl halide R—X (X representing a halogen):

Nitrocell-OH+R—X→Nitrocell-OR+HX

with X being a halogen chosen from chlorine, bromine and iodine.

reaction with an epoxide:

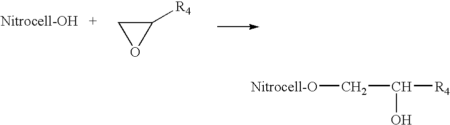

R₄ representing a chain forming part of the chain R defined above, the said chain R being represented herein by the group —CH₂—(CHOH)—R₄;

reaction with an aldehyde in reductive medium (such as triethylsilane in the presence of platinum):

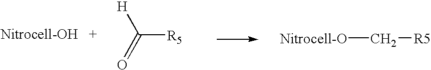

R₅ representing a chain forming part of the chain R, the said chain R being represented herein by the group —CH₂—R₅.

According to one variant, this reaction may proceed in two steps, the first step being a preliminary reaction of the aldehyde with a diol, such as glycol, to form a cyclic acetal:

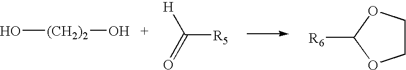

followed by a reaction of the cyclic acetal with the nitrocellulose:

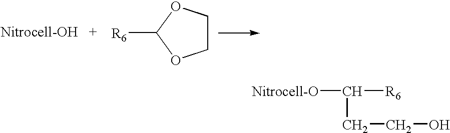

R₆ representing a chain forming part of the chain R, the said chain R being represented herein by the group —CH—(CH₂—CH₂—OH)—R₆.

reaction with a mixed ether R—O—R′ with R′ denoting a C₁-C₄ alkyl radical, in acidic medium:

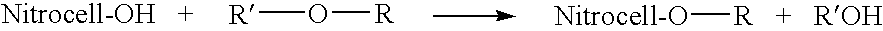

addition reaction of the free —OH groups of the nitrocellulose onto a double bond, for example a terminal double bond, borne by the radical to be grafted, in the presence of PdCl₂ and HgCl₂:

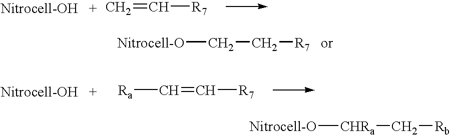

R₇ representing a chain forming part of the chain R, represented herein by the group —CH₂—CH₂—R₇ and Rₐ and R_{b} representing a chain forming part of the chain R, represented herein by the group —CHRₐ—CH₂—R_{b}.

Other etherification reactions may be envisaged, especially those mentioned in the book “Advanced Organic Chemistry”, J. March, John Wiley & Son, Edition 1992.

2) Esterification

Examples of esterification reactions that may be mentioned are the following, for which reactions Y represents a CO bonding group:

reaction with a carboxylic acid R—CO₂H:

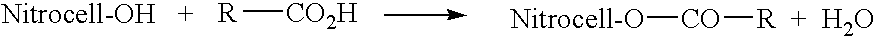

reaction with an acid chloride R—COCl or transesterification with an ester R—COOR′, such that:

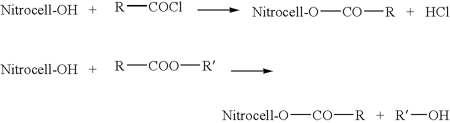

reaction with an acid anhydride, for example:

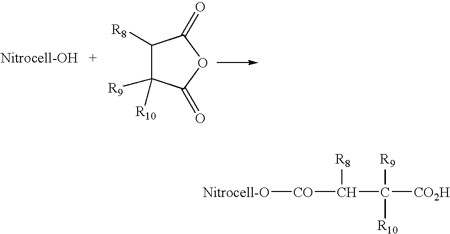

with R₈, R₉ and R₁₀ being such that —(CHR₈)—CR₉R₁₀—CO₂H represents R.

3) Transesterification with a Carbonate

The following reaction with a carbonate R′—O—CO—O—R may be envisaged, for which reaction Y represents a —CO—O— bonding group:

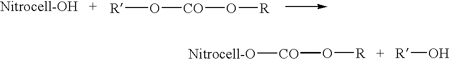

4) Esterification with a Sulphonyl Chloride

As examples of esterification reactions with a sulphonic acid or a sulphonyl chloride, for which reactions Y represents an —SO₂— bonding group, the following reaction may be envisaged:

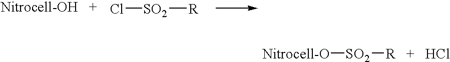

5) Reaction with an Isocyanate

As examples of reactions for forming carbamate bonds, for which reactions Y represents a —CO—NH— bonding group, the following reaction with an isocyanate OCN—R may be envisaged:

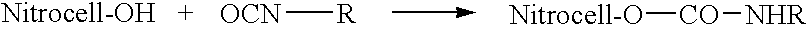

6) Reaction with an Alkoxysilane

As examples of reactions of nitrocellulose with an alkoxysilane, for which reactions Y represents a bonding group —Si(R₁₂)₂—, the following reaction may be envisaged:

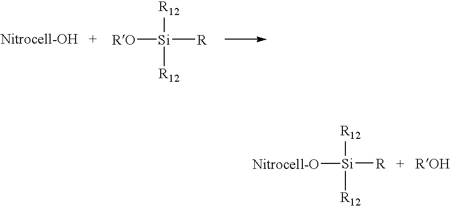

with the radicals R₁₂, which may be identical or different, possibly being a linear or branched hydrocarbon-based chain of 1 to 500 carbon atomd or a cyclic hydrocarbon-based chain of 3 to 500 carbon atoms, said chain being saturated or unsaturated and possibly comprising one or more O, N, S, Si and/or P atoms, preferably from 1 to 10 carbon atoms, the radicals R₁₂ thus having the same definition as the radicals R₃ defined above.

Given the general properties of nitrocellulose, the conditions required for the reactions for grafting a hydrocarbon-based chain of non-polymeric nature should preferably satisfy a certain number of criteria.

Specifically, the starting nitrocellulose is a hazardous reagent, due to its very high flammability and its explosive power, which imposes the need to adapt the reactions mentioned above, to make a preffered choice of reagents and to apply strict experimental conditions.

Thus, in order to obtain a suitable degree of grafting, it is preferable to work in a homogeneous solvent medium, the solvent or mixture of solvents needing to be chosen so as to be able simultaneously to dissolve the starting nitrocellulose, the grafting reagents and, preferably, the nitrocellulose modified after grafting the groups —R.

Furthermore, the solvent or mixture of solvents should be inert with respect to the grafting reaction. Specifically, since the grafting reaction takes place on the free —OH groups of the nitrocellulose, the mixture(s) of solvents should, preferably, themselves be free of —OH groups that might co-react with the grafting reagents, and free of groups other than —OH which might also react with the grafting reagents (for example such as solvents containing a labile hydrogen).

Taking into account what has been stated regarding the characteristics of the solvents, the solvents or mixtures of solvents that may be used for these reactions generally have a polarity, defined by the magnitude E_{T}(30) defined from the transition energy Z relative to the charge transfer peak in the UV spectrum of the complex between the iodide ion and the 1-methyl- or 1-ethyl-4-carbomethoxypyridinium ion, with an E_{T}(30) of 51.9 and 33.9, as mentioned in the book “Advanced Organic Chemistry” pages 357-361.

Among the solvents that may be envisaged, mention may be made of acetone, methyl ethyl ketone, ethyl acetate, butyl acetate, dimethylformamide, dimethyl sulphoxide, dimethylacetamide, pyridine, tetrahydrofuran, dioxane and toluene, and mixtures thereof.

Accordingly, given that one of the industrial forms for delivering nitrocellulose is a collodion wetted with about 35% alcohol, it will be preferable:

either to work with another industrial presentation of nitrocellulose, such as “chips”, comprising 80% of polymer and 20% of a plasticizer such as dibutyl phthalate;

or to work with a nitrocellulose directly in solution in a solvent, for example of the acetate type, such as ethyl acetate or butyl acetate;

or, finally, to work with the collodion, the said collodion being subjected before use to a step of solvent exchange, by means of a suitable laboratory or industrial technique, such as selective distillation, azeotropic distillation, liquid/liquid extraction or dialysis.

Preferably, the grafting reagent(s) should be inert with respect to any reaction on the nitric ester groups of the nitrocellulose, since the said nitrocellulose needs to be maintained in nature and in amount.

Finally, given the hazards involved in handling nitrocellulose, the preferred choice of the type of grafting reactions and of reagents will be made, according to the invention, from mild reactions and reagents, i.e.:

working at a reaction temperature from 0 to 80° C. and preferably from 20 to 60° C.;

working, preferably, in the absence of oxygen, to avoid any risk of explosion by contact of the nitrocellulose with an oxidizing agent;

where appropriate, in the presence of one or more catalysts that are suitable for the reaction to promote the mild conditions.

The reaction will preferably be performed under inert gas, such as argon, with, at the start of the reaction, less than 1 ppm of dissolved oxygen in the solvent or mixture of solvents used for the reaction.

As regards all these preliminary comments, the preferred reactions and the combined reagents, that satisfy the preferred conditions mentioned above, are chosen according to the invention from the following reactions:

esterification of the hydroxyl functions by means of a carboxylic acid in the presence of a coupling reagent such as dicyclohexylcarbodiimide or derivatives thereof;

Non-limiting examples of carboxylic acids that may be mentioned include butanoic acid, pentanoic acid, hexanoic acid (or caproic acid), heptanoic acid, octanoic acid (or caprylic acid), 2-ethylhexanoic acid, nonanoic acid, decanoic acid (or capric acid), neodecanoic acid (or versatic acid), undecanoic acid, dodecanoic acid, isononanoic acid, and also branched C₁₆-C₄₀ fatty acids or linear unsaturated C₁₆-C₄₀ fatty acids;

esterification of the hydroxyl functions by means of a carboxylic acid chloride;

For example, carboxylic acid chlorides that may be mentioned are the chlorides of the acids mentioned above.

esterification of the hydroxyl functions by means of a carboxylic acid anhydride;

For example, carboxylic acid anhydrides that may be mentioned are the anhydrides of the acids mentioned above.

reaction with an alkyl monoisocyanate;

For example, the monoisocyanates may be chosen from butyl isocyanate, isobutyl isocyanate, pentyl isocyanate, hexyl isocyanate, heptyl isocyanate, octyl isocyanate, 2-ethylhexyl isocyanate, nonyl isocyanate, decyl isocyanate, undecyl isocyanate and dodecyl isocyanate.

reaction with a monoalkoxysilane;

For example, the monoalkoxysilanes may be chosen from dimethylmethoxybutylsilane, dimethylmethoxypentylsilane, dimethylmethoxyhexylsilane, dimethylmethoxy(C₇ to C₁₂ alkyl)silane.

reaction with a monoepoxide, catalysed in acidic or basic medium or in the presence of alumina;

For example, the monoepoxide may be chosen from 1,2-epoxyhexane, 1,2-epoxyheptane, 1,2-epoxyoctane, 1,2-epoxynonane or 1,2-epoxyneodecane (sold by Shell under the brand name Cardura E).

reaction with an alkyl halide;

For example, the alkyl halide may be chosen from alkyl chlorides such as 1-chlorobutane, 1-chloropentane or 1-chlorohexane.

reaction with a C₁-C₄ alkyl monoacetal in acidic medium;

For example, alkyl monoacetals that may be mentioned include butyl methyl ether, pentyl methyl ether, hexyl methyl ether and dodecyl methyl ether.

addition reaction of the —OH functions of the nitrocellulose onto a reagent bearing a double bond.

As regards the operating conditions, the grafting reaction, using the reactions and reagents specified above, will preferably be performed by dissolving the nitrocellulose and the grafting reagents in an oxygen-free solvent or mixture of solvents. The nitrocellulose concentration in the solution is, for example, from 0.5% to 30% and preferably from 1% to 10%. The reaction temperature is raised from 0 to 80° C., depending on the reaction and the reagent chosen. Depending on the type of reaction, one or more catalysts may be added. The reaction progress is monitored by standard techniques such as IR, NMR or UV spectroscopy or thin layer chromatography (TLC). The product obtained is then purified and analysed by standard purification and analysis techniques.

According to a second embodiment of the process, corresponding to the case in which the hydrocarbon-based chain R is of polymeric origin, the grafting reaction of the said chain may be performed in different ways.

Thus, according to a first embodiment, the grafting reaction may consist in reacting directly on all or some of the free hydroxyl functions of the starting nitrocellulose, the reactive ends of polymers, the chains of which form the hydrocarbon-based chain R.

The grafting reaction may be represented by the following overall reaction:

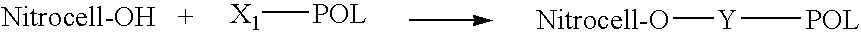

with POL representing the polymer whose sequence corresponds to the definition of the hydrocarbon-based chain R given above, X₁ representing a function borne by the polymers, the said function being reactive towards hydroxyls of the nitrocellulose and Y corresponding to the same definition as that given above and resulting from the reaction of —OH with X₁.

In the same respect as for the grafting of hydrocarbon-based chains of non-polymeric nature, the functions X₁ that are reactive with respect to the free hydroxyl functions of the nitrocellulose may be chosen from epoxide, aldehyde, acetal, halogen (chlorine, bromine or iodine), ethylenic, carboxylic acid or derivative (chloride, anhydride or C₁-C₄ alkyl ester), carbonate, sulphonic acid or sulphonyl chloride, isocyanate and monoalkoxysilane functions.

However, given the hazards involved in handling the nitrocellulose mentioned above, the reactive functions X₁ of the hydrocarbon-based polymers employed in the grafting reaction are preferably chosen from carboxylic acid chloride, carboxylic acid anhydride, carboxylic acid (in the presence of a coupling reagent of the DCCI type), monoisocyanate, monoalkoxysilane, monoepoxide, halogen (Cl, Br or I), C₁-C₄ alkyl monoether, or vinylic double bond type functions.

The starting polymers POL-X₁ need to be synthesized, apart from those for which X₁ is a reactive double bond of vinyl type, many of which are commercially available. The polymers POL-X₁ may be synthesized, for example, starting with a polymer comprising a reactive function other than X₁, which is converted via standard reactions into a suitable X₁.

An example that may be mentioned is the following reaction:

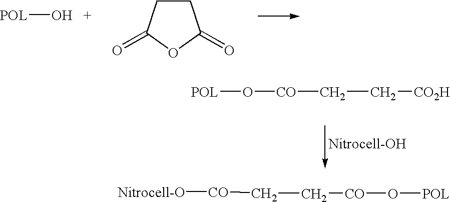

According to a second embodiment, the grafting reaction may consist, in a first stage, in converting all or some of the hydroxyl functions of the starting nitrocellulose into reactive functions, and then, in a second stage, in reacting the said reactive functions with the appropriate reactive ends of polymers comprising the said hydrocarbon-based chain R.

An example that may be mentioned is the following reaction:

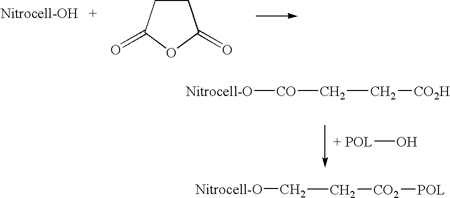

The reaction of Nitrocell-OCOCH₂CH₂—CO₂H with Nitrocell-OH may take place, but conditions are established in which this reaction is minimized (crosslinking is avoided).

As regards the nature of the polymer that may be employed in the grafting reaction with nitrocellulose, to make the hydrocarbon-based chains R, this polymer may be of any chemical nature (vinyl polymers, polycondensates such as polyesters, polyamides, polyurethanes, polyurea, etc.), provided that the said polymer satisfies the definition of the hydrocarbon-based chain R defined above.

Such polymers may be chosen, for example, from vinyl polymers and polycondensates.

According to the invention, the vinyl polymers denote both oligomers and polymers derived from the homopolymerization or copolymerization of vinyl monomers in the broad sense, i.e. (meth)acrylic, styrene, allylic monomers.

Examples of vinyl polymers that may be mentioned include the polymers obtained by homopolymerization or copolymerization of monomers chosen from:

alkyl acrylates with a linear or branched C₁-C₃₀ alkyl group, such as methyl acrylate, ethyl acrylate, propyl acrylate, isopropyl acrylate, n-butyl acrylate, isobutyl acrylate, n-pentyl acrylate, n-hexyl acrylate, n-heptyl acrylate, n-octyl acrylate, 2-ethylhexyl acrylate, nonyl acrylate, decyl acrylate, undecyl acrylate and dodecyl acrylate;

alkyl methacrylates with a C₆-C₃₀ alkyl group or a C₁-C₅ alkyl group, in the case of copolymers, provided that the glass transition temperature satisfies the condition mentioned below;

vinyl esters, with a linear or branched C₃-C₃₀ alkyl group, such as vinyl propionate, vinyl neodecanoate, vinyl octanoate, vinyl decanoate, vinyl laurate or a C₁-C₂ alkyl group, in the case of copolymers, provided that the glass transition temperature of the said copolymer satisfies the condition mentioned below;

vinyl ethers with a C₃-C₃₀ alkyl group, such as propyl vinyl ether, butyl vinyl ether, isobutyl vinyl ether, tert-butyl vinyl ether, pentyl vinyl ether, hexyl vinyl ether, octyl vinyl ether, 2-ethylhexyl vinyl ether, nonyl vinyl ether, decyl vinyl ether and dodecyl vinyl ether;

α-olefins, for example C₂ to C₂₀, in particular α-olefin copolymers whose monomers give crystalline homopolymers; and homopolymers or copolymers of branched α-olefins. Mention is made in particular of isobutylene homopolymer and of copolymers between ethylene (or propylene) and longer α-olefins such as butene, hexene, octene, decene or dodecene. For these α-olefins, mention may also be made of the non-crystalline copolymers thereof with cycloolefins, in particular the copolymers between ethylene (or propylene) and norbornene or norbornene derivatives;

dienes, for example C₄ to C₂₀ dienes, such as butadiene, isoprene, hexadiene, etc., giving copolymers with other vinyl monomers such as those mentioned above, and more particularly with styrene or substituted styrenes.

As mentioned previously, the hydrocarbon-based chains R grafted onto the nitrocellulose may comprise one or more adhesion-promoting groups. These adhesion-promoting groups may be introduced by copolymerization with suitable vinyl monomers, such as:

monomers bearing —CO₂H groups, such as (meth)acrylic acid, crotonic acid, maleic acid, itaconic acid or allyloxyacetic acid;

monomers bearing —OH groups, such as allyl alcohol, 2-hydroxyethyl (meth)acrylate or 2-hydroxypropyl (meth)acrylate;

monomers bearing amide groups, such as (meth)acrylamide, N-alkyl(meth)acrylamide with identical or different C₁-C₁₂ alkyls, N-vinylacetamide or N-vinylformamide;

monomers bearing pyrrolidono groups, such as N-vinylpyrrolidone;

monomers bearing amine groups, such as vinylamine, allylamine or N,N-dimethylaminoethyl (meth)acrylate;

monomers bearing oxazoline groups, such as N-vinyloxazoline;

monomers bearing pyridine groups, such as N-vinylpyridine;

monomers bearing chlorine groups, such as vinyl chloride, vinylidene chloride or chloroolefins;

monomers bearing acetoacetyl groups, such as 2-acetoacetoxy(meth)acrylate;

monomers bearing acetal groups, such as (meth)acrylamidobutyraldehyde dimethyl acetal or (meth)acrylamidoacetaldehyde dimethyl acetal;

monomers bearing siloxane groups, such as 3-(trimethoxysilyl)propyl methacrylate.

Examples of polycondensates that may be mentioned include:

polyesters, such as those derived from the polyesterification between diols and diacids, such as ethylene glycol, propylene glycol, diethylene glycol, neopentyl glycol, 1,4-butanediol, furandimethanol, cyclohexanedimethanol, glycerol, trimethylolpropane and pentaerythritol, and mixtures thereof, with polycarboxylic acids, in particular carboxylic acids and the C₁-C₄ ester derivatives thereof, for example succinic acid, glutaric acid and adipic acid or the dimethyl esters thereof, phthalic anhydride or dimethyl terephthalate, or with lactones, for example caprolactone;

polyesteramides obtained by inclusion of amino alcohols such as ethanolamine in polyesterification mixtures;

polycarbonates, obtained by reacting diols such as 1,3-propanediol, 1,4-butanediol, 1,8-hexanediol, diethylene glycol or tetraethylene glycol with diaryl, diacyl or aliphatic carbonates, for example diphenyl carbonate, or with phosgene;

polyamides obtained by condensation between an aliphatic, cycloaliphatic or aromatic C₃-C₅₀ carboxylic diacid (or C₁-C₄ ester derivative) and a linear or branched aliphatic, cycloaliphatic or aromatic C₂-C₅₀ diamine, the diacids possibly being chosen from the diacids mentioned above, the diamines possibly being chosen from ethylenediamine, 1,2-diaminopropane, 1,3-diaminopropane, 1,4-diaminobutane, 1,2-diamino-2-methylpropane, 1,6-diaminohexane, 1,10-diaminodecane, isophoronediamine, adamantanediamine and 2,6-diaminopyridine; the polyurethanes, polyureas and polyurea-urethanes obtained by polyaddition between C₄-C₁₀₀, preferably C₄-C₃₀, aliphatic, cycloaliphatic and/or aromatic diisocyanates, such as hexamethylene diisocyanate, isophorone diisocyanate, toluene diisocyanate or diphenylmethane diisocyanate, and diols such as those defined above, or diamines such as those defined above, or diol/diamine mixtures;

polyethers such as copolymers between oxyethylene and oxypropylene, and polytetramethylene oxide.

As regards the introduction of the adhesion-promoting groups into polycondensates of this type, these groups are introduced directly by means of monomers including these groups.

As non-limiting examples, for introducing —CO₂H groups, dimethylolpropionic acid may be used (in particular for polyurethanes and polyureas); for introducing amino groups, N,N-dimethylethanolamine may be used (in particular for polyureas).

The polymers and polycondensates containing a group that is reactive towards the free hydroxyl functions of the nitrocellulose may be prepared in different ways.

Thus, for the polymers bearing a reactive group, a first variant consists in preparing the said polymers by polymerization of the selected monomer(s) in the presence of a transfer agent bearing a reactive group.

The polymer thus formed comprises two ends, i.e. an α end comprising a polymerization-initiating residue, and an ω end comprising the group derived from the transfer agent.

The polymerization conditions and the choice of transfer agent are preferably such that the chain-terminating reaction takes place by transfer.

For example, to prepare a poly(2-ethylhexyl acrylate) telomer with a reactive end group and a molecular weight of 1 150, the process may be performed in two steps as described in international patent application WO-A-97/35541.

In a first step, 2-ethylhexyl acrylate is polymerized by initiation with azobisisobutyronitrile, with mercaptoethanol as the transfer agent, to give a polymer with an —OH end.

In a second step, the —OH end that is not reactive towards the free OH— groups of the nitrocellulose is converted into a reactive group, for example by reaction with a cyclic anhydride, such as succinic anhydride, under conditions such that the double esterification cannot take place.

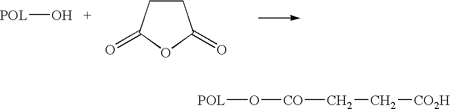

To obtain directly a reactive group —CO₂H directly, mercaptopropionic acid may be used as transfer agent, in order to obtain the polymer containing a POL-S—CH₂—CH₂—CO₂H reactive end.

Other polymers ending with a vinyl end group (in the broad sense, including (meth)acrylic, styrene or allylic) are commercially available and may accordingly be used directly in the nitrocellulose-grafting reaction. Such oligomers are sold in particular by the Japanese company Toa Gosei.

As regards the polycondensates containing a reactive end group X₁, their preparation, especially as regards the polyesters and polyamides, does not require any particular arrangement for the introduction of the reactive group, since this group is already present at the end of the chain.

For example, a polyester generally comprises, at the end of preparation, a reactive —CO₂H end group and an —OH end group. It should be noted that this —OH end group will preferably be blocked with an inert group lacking labile hydrogen, so as not to interfere with the grafting reaction on the nitrocellulose.

The same comments apply to a polyamide, which has a reactive —CO₂H end group and an —NH₂ end group to be protected with a group that is inert with respect to the grafting reaction with the nitrocellulose.

A reactive group X₁ may also be introduced into the polycondensate, by introducing into the reaction medium, during the polycondensation, a reagent bearing the group X₁, which must be inert with respect to the type of polycondensation chosen or inert under the experimental conditions of the polycondensation, and a single group capable of participating in the polycondensation. This reagent is therefore monofunctional with respect to the polycondensation and thus acts as a chain limiter.

This reagent that is monofunctional with respect to the polycondensation and bears a group that is reactive with respect to hydroxyl functions of the nitrocellulose is preferably introduced during the polycondensation, in order for the chains of the polymer to be terminated with only one reactive group Xᵢ.

According to a third embodiment for grafting hydrocarbon-based chains R of polymeric nature onto the free hydroxyl functions of a nitrocellulose, the grafting reaction consists, in a first stage, in converting all or some of the free hydroxyl functions of the starting nitrocellulose into functions that are capable of acting as co-initiator for a vinylic polymerization reaction, and then, in a second stage, in reacting the said converted nitrocellulose with vinyl monomers.

This embodiment may be carried out by using, in particular, the novel types of “living” controlled free-radical polymerization, such as the “Atom Transfer Polymerization” (ATRP) process. This process requires the prior conversion of the free —OH groups of the nitrocellulose into groups bearing a halogen atom (chlorine or bromine), the said groups acting as co-initiators and grafting sites for an initiated vinyl ATRP polymerization with a Cu(I) salt system or a Cu(I)/bipyridine salt system, as mentioned in patent applications WO 97/18247 and WO 98/51722.

Such a process for modifying the —OH groups followed by initiation of an ATRP polymerization in the presence of Cu(I) is described in patent applications WO 071 606 and WO 071 607 and in the article by M M. Guerrini, Macromol. Rapid. Commun., 21, No. 10, pages 1-6 (2000). Thus, the nitrocellulose-grafting process, according to an ATRP process, is as follows:

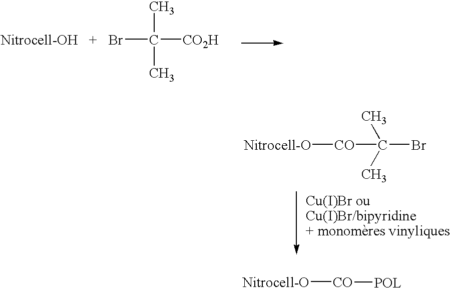

POL representing the polymer derived from the polymerization of the vinyl monomers, such as the alkyl (meth)acrylates.

The polymers that may be used as grafts for the nitrocellulose according to the invention preferably have a glass transition temperature of less than or equal to 60° C., especially ranging from −150° C. to 60° C., preferably less than or equal to 40° C., especially ranging from −150° C. to 40° C., or a melting point of less than or equal to 60° C., preferably less than or equal to 40° C. The glass transition temperature and melting point values may be determined in a known manner, by the glass transition temperature or melting point values of the monomers, which are, for example, those that may be found in a reference manual such as the Polymer Handbook, 3rd Edition, 1989, John Wiley.

Alternatively, the nitrocellulose modified according the invention can be prepared by carrying out the grafting reaction of chains R, as defined above, on one part of free hydroxyl functions of a starting cellulose to obtain a cellulose, the free hydroxyl groups of which are replaced by —O—Y—R, as defined above, followed by a complete or partial nitration of remaining free hydroxyl groups to obtain the desired modified nitrocellulose.

The invention will now be described with reference, to the following examples, which are given as non-limiting illustrations.

### EXAMPLES

The following examples illustrate the preparation of modified nitrocelluloses according to the present invention, and also examples of formulations comprising such nitrocelluloses. However, they are not limiting of the invention.

### Example 1

### Preparation of a Modified Nitrocellulose According to the Invention

In this example, a modified nitrocellulose is prepared, comprising radicals —OYR, replacing some of the free hydroxyls of the starting nitrocellulose, Y being a —C(C═O)—NH— bonding group and R being a —(CH₂)₇—CH₃ chain.

122 g of nitrocellulose (reference NCE20 from Bergerac, containing 18% by weight of dibutyl phthalate) are dissolved in 1 litre of an anhydrous mixture of methyl ethyl ketone/toluene (60/40 v/v), with stirring, in a three-necked flask on which is mounted a condenser. The solution is degassed three times and placed under nitrogen. 24.75 g of octyl isocyanate are added to the reaction medium with continued stirring, and in the presence of argon. After addition of a catalytic amount of dibutyltin dilaurate, the solution is heated to 50° C. and maintained at this temperature for 48 hours. After cooling to room temperature, the reaction medium is placed in contact with 5 litres of water, in which the modified nitrocellulose precipitates. This precipitate is recovered and washed 3 times with a large excess of water. It is then back-dialysed in acetone for 5 days, in order to remove the impurities and the dibutyl phthalate initially present in the nitrocellulose. After the dialysis, the acetone is evaporated off in a controlled manner until the final amount corresponds to a composition of 50% acetone to 50% polymer.

1 g of the solution obtained is dried at 50° C., until the solvent has been completely evaporated off. The polymer film thus obtained is analysed by infrared. A band appears at 2255 cm⁻¹, corresponding to the presence of the carbamate bond between the hydroxyl functions of the nitrocellulose and the isocyanate function of the octyl isocyanate.

The polymer thus obtained has a final composition (by weight) of 65% modified units to 35% unmodified units.

### Example 2

A coloured nail varnish having the composition below is prepared:

| | | |
|---|---|---|
| modified nitrocellulose of Example 1: | 20 | g |
| plasticizer: | 0 to 20 | g |
| additional film-forming polymer: | 0 to 20 | g |
| gelling agent: | 0 to 10 | g |
| alcohol: | 0 to 15 | g |
| dyestuffs: | 0 to 10 | g |
| solvents (mixture of butyl acetate | qs 100 | g |
| and ethyl acetate): | | |

The contents of plasticizers and of additional film-forming polymer are adapted to the predefined mechanical criteria of the film.

The nail varnish applies correctly to the nails, and the film obtained is glossy and has good staying power after 5 days without wear or chipping.

### Example 3

A nail varnish having the composition below is prepared:

| | | |
|---|---|---|
| modified nitrocellulose of Example 1: | 20 | g |
| N-ethyl-o,p-toluenesulphonamide: | 10 | g |
| hectorite: | 1.7 | g |
| DC Red 34 red pigment: | 1.5 | g |
| solvents (ethyl acetate, | qs 100 | g |
| butyl acetate): | | |

The varnish applies easily to nails. The makeup film adheres well to the nails and shows good staying power, and especially good resistance to chipping.

### Example 4

A nail varnish having the composition below is prepared:

| | | |
|---|---|---|
| modified nitrocellulose of Example 1: | 20 | g |
| acetyl tributyl citrate: | 8 | g |
| hectorite: | 1.7 | g |
| DC Red 34 red pigment: | 1.5 | g |
| solvents (ethyl acetate, butyl acetate): | qs 100 | g |

The varnish applies easily to the nails and the makeup obtained is glossy, adheres well to the nails and has good resistance to chipping.

In this application every numerical limit and range specifically include therewithin all values and subranges as if specifically written out.

All references, texts, documents, patents, publications, applications, standards, tests, etc. mentioned herein are incorporated by reference in their entirety.

1. A composition comprising an organic solvent and a nitrocellulose comprising free hydroxyl functions replaced, totally or partially, with one or more radicals —OYR, in which R represents a linear or branched hydrocarbon-based chain of 1 to 500 carbon atoms or a cyclic hydrocarbon-based chain of 3 to 500 carbon atoms, said chains being saturated or unsaturated and optionally comprising one or more of O, N, S, Si and/or P atoms, and Y represents a single bond or a bonding group.

2. The composition according to claim 1, in which R represents a hydrocarbon-based chain linked to oxygen atoms of anhydroglucose rings of the nitrocellulose via bonding groups Y, wherein Y is selected from the group consisting of —(C═O)—, —(C═O)O—, —SO₂, —CO—NH—, —CO—NR′— and —Si(R₃)₂— groups, R₃, which may be identical or different, being a linear or branched hydrocarbon-based chain of 1 to 500 carbon atoms or a cyclic hydrocarbon-based chain of 3 to 500 carbon atoms, said chains being saturated or unsaturated and optionally comprising one or more of O, N, S, Si and/or P atoms and R′ denoting a C1 to C4 alkyl radicals.

3. The composition according to claim 1, in which the hydrocarbon-based and cyclic hydrocarbon-based chains R contain from 4 to 100 carbon atoms.

4. The composition according to claim 1, in which the hydrocarbon-based and cyclic hydrocarbon-based chains R comprise at least one adhesion-promoting group.

5. The composition according to claim 4, in which the at least one adhesion-promoting group is located in the hydrocarbon-based chains R laterally and/or at the end thereof.

6. The composition according to claim 4, in which the at least one adhesion-promoting group is present in an amount of less than or equal to 10% by weight of the nitrocellulose.

7. The composition according to claim 4, in which the at least one adhesion-promoting group is selected from the group consisting of:
hydroxyl —OH;
carboxylic acid or ester —CO₂R₁;
chloro —Cl;
amino —NR₁R₂ with R₁ and R₂, which may be identical or different;
pyridine of formula: 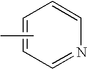
pyrimidino of formula: 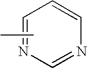
oxazolino of formulae: 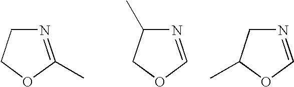
amido —NH—CO—R′ or —CO—NH—R₁;
pyrrolidono of formulae: 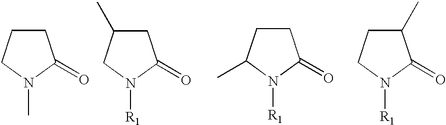
carbamoyl —O—CO—NH—R′ or —NH—CO—O—R′;
thiocarbamoyl of formula —O—CS—NHR₁ or —NH—CS—O—R′;
carbonato —O—CO—O—R′;
ureyl —NR₁—CO—N(R₁)₂, the radicals R₁ being identical or different;
thioureyl —NR₁—CS—N(R₁)₂, the radicals R₁ being identical or different;
oxamido —NR₁—CO—CO—N(R₁)₂, with the radicals R₁ being identical or different;
guanidino —NH—C(═NH)—N(R₁)₂, with the radicals R₁ being identical or different;
biguanidino —NH—C(═NH)—NH—C(═NH)—N(R₁)₂, with the radicals R₁ being identical or different;
sulphonic ester —O—S(═O)₂—R′;
sulphonamido —NR₁—S(═O)₂—R′;
acetoacetyl —O—CO—(CH₂)ₐ—CO—R′, with a denoting an integer ranging from 1 to 10 and preferably ranging from 1 to 5;
siloxane —Si—(R′)_{3-b}(OR′)_{b}, with b denoting an integer ranging from 1 to 3;
acetal —OR′,
R₁ and R₂ denoting H or a C1 to C4 alkyl radical, R′ denoting a C1 to C4 alkyl radical.

8. The composition according to claim 1, in which a degree of molar grafting of the radicals of formula OYR replacing, totally or partially, the free hydroxyl groups per anhydroglucose ring of the nitrocellulose is from 0.1 to 2.

9. The composition according to claim 1, in which R is linked to the oxygen atoms of anhydroglucose rings of the nitrocellulose via single bonds.

10. The composition according to claim 4, wherein the at least one adhesion-promoting group is present in an amount less than or equal to 5% by weight of the nitrocellulose.

11. The composition according to claim 1, in which, when R is of polymeric nature, they are not vinylic and/or acrylic polymers.

12. The composition according to claim 1, in which the nitrocellulose has only one glass transition temperature Tg such that Tg is less than or equal to 100° C.

13. The composition according to claim 1, in which the nitrocellulose is capable of forming a film that has a storage modulus E′ of greater than or equal to 100 MPa and/or a damping power tgδ greater than or equal to 0.4, at a temperature of 30° C. and at a frequency of 1 Hz.

14. The composition according to claim 1, in which the nitrocellulose is capable of forming a film that has an ultimate strain εᵣ of greater than or equal to 5% and/or an energy at failure per unit of volume Wᵣ of greater than or equal to 0.2 J/cm³.

15. The composition according to claim 1, in the form of a nail varnish for making up the nails and/or false nails.

16. The composition according to claim 1, in which the content of modified nitrocellulose is from 0.1% to 60% by weight relative to the total weight of the composition.

17. The composition according to claim 1, in which the organic solvent is selected from the group consisting of ketones that are liquid at room temperature, alcohols that are liquid at room temperature, glycols that are liquid at room temperature, propylene glycol ethers that are liquid at room temperature, esters containing from 3 to 8 carbon atoms in total, ethers that are liquid at room temperature, alkanes that are liquid at room temperature, cyclic aromatic compounds that are liquid at room temperature, aldehydes that are liquid at room temperature, and mixtures thereof.

18. The composition according to claim 1, further comprising one or more additives selected from the group consisting of non-nitrocellulose film-forming polymers, plasticizers, dyestuffs thickeners, spreading agents, wetting agents, dispersants, antifoams, preserving agents, UV-screening agents, active agents, surfactants, waxes, moisturizers, fragrances, neutralizers, stabilizers and antioxidants.

19. Nitrocellulose comprising free hydroxyl functions replaced, totally or partially, with radicals —OYR, in which R represents a linear or branched hydrocarbon-based chain of 1 to 500 carbon atoms or a cyclic hydrocarbon-based chain of 3 to 500 carbon atoms, said chains being saturated or unsaturated and optionally comprising one or more O, N, S, Si and/or P atoms, and Y represents a single bond or a bonding group, and said R chain comprising at least one adhesion-promoting group selected from the group consisting of:
chloro —Cl;
amino —NR₁R₂ with R₁ and R₂, which may be identical or different;
pyridine of formula: 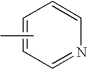
pyrimidino of formula: 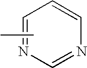
oxazolino of formulae: 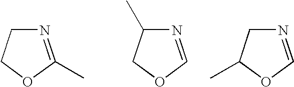
amido —NH—CO—R′ or —CO—NH—R₁;
pyrrolidono of formulae: 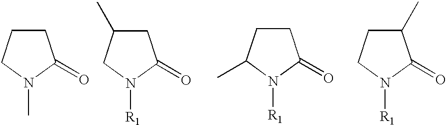
carbamoyl —O—CO—NH—R′ or —NH—CO—O—R′;
thiocarbamoyl of formula —O—CS—NHR₁ or —NH—CS—O—R′;
carbonato —O—CO—O—R′; —ureyl —NR₁—CO—N(R₁)₂, the radicals R₁ being identical or different;
thioureyl —NR₁—CS—N(R₁)₂, the radicals R₁ being identical or different;
oxamido —NR₁—CO—CO—N(R₁)₂, with the radicals R₁ being identical or different;
guanidino —NH—C(═NH)—N(R₁)₂, with the radicals R₁ being identical or different;
biguanidino —NH—C(═NH)—NH—C(═NH)—N(R₁)₂, with the radicals R₁ being identical or different;
sulphonic ester —O—S(═O)₂—R′;
sulphonamido —NR₁—S(═O)₂—R′; —acetoacetyl —O—CO—(CH₂)ₐ—CO—R′, with a denoting an integer ranging from 1 to 10 and preferably ranging from 1 to 5;
siloxane —Si—(R′)_{3-b}(OR′)_{b}, with b denoting an integer ranging from 1 to 3;
acetal —OR′,
R₁ and R₂ denoting H or a C1 to C4 alkyl radical, R′ denoting a C1 to C4 alkyl radical.

20. Nitrocellulose according to claim 19, in which R is linked to the oxygen atoms of anhydroglucose rings of the nitrocellulose via bonding groups Y selected from the group consisting of —(C═O)—, —(C═O)O—, —SO₂, —CO—NH—, —CO—NR′— and —Si(R₃)₂— groups, the radicals R₃, which may be identical or different, being a linear or branched hydrocarbon-based chain of 1 to 500 carbon atoms or a cyclic hydrocarbon-based chain of 3 to 500 carbon atoms, said chains being saturated or unsaturated and optionally comprising one or more O, N, S, Si and/or P atoms and R′ denoting a C1 to C4 alkyl radical.

21. Nitrocellulose according to claim 19, in which the R contains from 4 to 100 carbon atoms.

22. Nitrocellulose according to claim 19, in which the at least one adhesion-promoting group is located in the hydrocarbon-based chains R laterally and/or at the end thereof.

23. Nitrocellulose according to claim 19, in which the at least one adhesion-promoting group is present at a content of less than or equal to 10% by weight.

24. Nitrocellulose according to claim 19, in which a degree of molar grafting of the radicals of formula OYR replacing, totally or partially, the free hydroxyl groups per anhydroglucose ring of the nitrocellulose is from 0.1 to 2.

25. Nitrocellulose according to claim 19, in which the R is linked to the oxygen atoms of anhydroglucose rings of the nitrocellulose via single bonds.

26. Nitrocellulose according to claim 19, in which the at least one adhesion-promoting group is present at a content of less than or equal to 5% by weight.

27. Nitrocellulose according to claim 19, in which the nitrocellulose has only one glass transition temperature Tg such that Tg is less than or equal to 100° C.

28. Nitrocellulose according to claim 19, in which the nitrocellulose is capable of forming a film that has a storage modulus E′ of greater than or equal to 100 MPa and/or a damping power tgδ greater than or equal to 0.4, at a temperature of 30° C. and at a frequency of 1 Hz.

29. Nitrocellulose according to claim 19, in which the nitrocellulose is capable of forming a film that has an ultimate strain εᵣ of greater than or equal to 5% and/or an energy at failure per unit of volume Wᵣ of greater than or equal to 0.2 J/cm³.

30. Nitrocellulose according to claim 19, in which the nitrocellulose has only one glass transition temperature Tg such that Tg is from 40 to 60° C.

31. Nitrocellulose according to claim 19, in which the nitrocellulose is capable of forming a film that has a storage modulus E′ of greater than or equal to 300 MPa and/or a damping power tgδ greater than or equal to 0.6, at a temperature of 30° C. and at a frequency of 1 Hz.

32. Nitrocellulose according to claim 19, in which the nitrocellulose is capable of forming a film that has an ultimate strain εᵣ of greater than or equal to 15% and/or an energy at failure per unit of volume Wᵣ of greater than or equal to 1 J/cm³.

33. A process for preparing the nitrocellulose as defined according to claim 19, comprising grafting directly on all or some of the free hydroxyl functions of a starting nitrocellulose, the reactive end groups of molecules whose chains constitute the hydrocarbon-based chain R.
